(19) United States
(12) Patent Application Publication
(10) Pub. No.: US 2014/0171837 A1
(43) Pub. Date: Jun. 19, 2014

(54) VACUUM CAST ("VAC-CAST") AND METHODS FOR TREATMENT OF PLANTAR WOUNDS

(71) Applicant: Karen Aiko Harcourt, Lebanon, PA (US)

(72) Inventor: Karen Aiko Harcourt, Lebanon, PA (US)

(21) Appl. No.: 13/858,915

(22) Filed: Apr. 8, 2013

Related U.S. Application Data
(60) Provisional application No. 61/739,009, filed on Dec. 18, 2012.

(51) Int. Cl. A61F 5/058

(52) U.S. Cl. CPC A61F 5/05833
USPC 601/7

(57) ABSTRACT

A combination of four wound care products include (1) a wound vacuum dressing providing a more sterile cover over the wound to prevent infection, provide a closed system to prevent maceration and soiling from drainage to the surrounding skin and peg insole pad, to introduce and maintain negative pressure, to decrease excessive fluid to the wound(s) and provide an environment to increase granulation and epithelialization; (2) a compression hose for additional support to the lower leg, ankle, and foot, aiding in proper fluid pressure control in the lower extremity and protects against friction of the contact cast against the lower extremity; (3) a removable peg insole foot insert offloading pressure and friction from the wound as well as providing a pressure free pathway to the ankle connection point which prevents obstruction, and (4) a total contact cast with inner padding layer to further off load pressure from the wound(s).

### CROSS-REFERENCE TO RELATED APPLICATION

This application claims the benefit of priority of U.S. provisional application No. 61/739,009, filed Dec. 18, 2012, the contents of which are herein incorporated by reference.

### BACKGROUND OF THE INVENTION

The present invention relates to plantar wound dressings, more particularly, to the use of a vacuum dressing in combination with a compression stocking, a pressure off loading foot pad including a removable peg insole, and a pressure off loading cast (more commonly called a total contact cast), which is useful in the treatment of plantar wounds.

Patients with plantar wounds (wounds on the bottom of the foot) often have trouble healing for a variety of reasons; (1) Many patients also suffer from secondary conditions such as Diabetes, venous insufficiency, arterial insufficiency, and immune system deficiencies; (2) Patients often continue to walk, more or less, on the injured foot or feet, causing increased pressure, friction, and irritation to the wound(s); (3) Plantar wounds are subject to weight bearing loads interfering with granulation and epithelialization (formation of new cells), especially in patients that are overweight, or have a foot deformity such as charcot arthropathy (foot deformity from diabetes), pes cavis (high arch), bunion, etc.; (4) Plantar wounds come into contact with non-sterile surfaces and sources of infection, especially bacteria, due to their location on the bottom of the foot; (5) Fluid and fluid pressure build up is also more pronounced in plantar wounds than in wounds located on other parts of the anatomy, due to the fluid being pulled down from gravity, then exiting through the path of least resistance (the plantar wound). This fluid macerates the skin, weakening it and preventing granulation and epithelialization from occurring. (6) Patients with plantar wounds commonly suffer from impaired sensation at the wound site, called neuropathy, so they don't feel sensual indications of a worsening condition.

Current solutions for the treatment of plantar wounds in ambulatory patients do not adequately address the problems associated with secondary conditions, pressure, friction, exposure to infection, and fluid build-up.

There is a great need for an improved method and device for increasing healing rates and outcomes associated with ambulatory patients suffering from plantar wounds. This need is evidenced by the significant proportion of wound care patients with secondary conditions whom also suffer from chronic plantar wounds, and in particular the proportion of those chronic cases requiring amputation. Furthermore, clinical trials of the products and methods, documented herein, resulted in desired outcomes and accelerated rates of healing for patients who failed previously to respond to existing treatments and methods and in several cases the prognosis, prior to the new experimental treatment described herein, was amputation.

### SUMMARY OF THE INVENTION

In one aspect of the present invention, a vacuum cast comprises a layered wound vacuum dressing applied to plantar wounds of a patient; a vacuum connector applied to an outer sealed vacuum layer of the layered wound vacuum dressing and placed over the posterior Achilles tendon; extraction tubing extending from the vacuum tubing to a vacuum device; a peg insole applied to a sole of a foot of the patient, the peg insole having one or more wound cavities corresponding to a location of the plantar wounds, and a channel interconnecting the one or more wound cavities with a posterior portion of the foot; and a pressure cast layer applied about a lower leg and foot of the patient.

In another aspect of the present invention, a method for protecting and encouraging healing of plantar wounds comprises applying a layered wound vacuum dressing to plantar wounds of a patient, to reduce pressure, introduce negative pressure, and protect from friction, exposure to bacteria, and provide a conduit for the drainage of fluid and fluid pressure; locating and positioning a connector and extraction tubing to a sealed vacuum layer of the layered wound vacuum dressing, the connector being located over a posterior ankle in a low profile concavity of the Achilles tendon such that no compression of the connector and extraction tubing takes place; extending all inner and outer layers of the layered wound vacuum dressing from over an area of the wound to underneath an area of the connector and extraction tubing, forming a continuous and contiguous permeable conduit; applying a compression hose over a lower leg and the layered wound vacuum dressing, starting from below a knee and extending to just behind toes of the patient; applying an adjustable foot sole insole on the bottom of the foot of the patient, the insole having pegs removed over one or more wound cavities and a channel of pegs removed to form a pathway to the wound vacuum connector for additional offloading and pressure redirection; applying a pressure cast layer around the calf, ankle, and foot of the patient, the pressure cast layer covering the layered wound vacuum dressing and the insole to off load weight bearing pressure while allowing the connector and extraction tubing to protrude and project through the pressure cast layer un-impinged; and applying a vacuum device to the surgical tubing to create and maintain negative pressure on the wound and provide a conduit for removal of fluids, bacteria, and fluid pressure.

These and other features, aspects and advantages of the present invention will become better understood with reference to the following drawings, description and claims.

### BRIEF DESCRIPTION OF THE DRAWINGS

FIG. 1 is a side view of a vacuum cast according to an exemplary embodiment of the present invention;

FIG. 2 is a cross sectional view taken along line 2-2 of FIG. 1;

FIG. 3 is an exploded view of a portion of a vacuum cast, illustrating application of multiple layers thereof;

FIG. 4 is a rear detail perspective view showing a portion of the vacuum cast of FIG. 1, illustrating application of a sealed wound vacuum dressing outer layer and its required placement over the Achilles Tendon;

FIG. 5 is a side perspective view showing a portion of the vacuum cast of FIG. 1, illustrating application of a pressure cast padding layer as well as the vacuum layer and proper placement of the connector over the Achilles tendon;

FIG. 6 is a side perspective view showing a portion of the vacuum cast of FIG. 1, illustrating application of a peg sole and its required drainage pathway;

FIG. 7 is a rear and bottom view showing a portion of the vacuum cast of FIG. 1, illustrating application of a peg sole with the required drainage pathway and wrapping of the pressure cast;

FIG. 8 is a detailed perspective view of a portion of the vacuum cast of FIG. 1, illustrating mounting of a drainage reservoir and the vacuum device; and

FIG. 9 is a flow chart describing the required application process and sequence of the vacuum cast of the present invention.

### DETAILED DESCRIPTION OF THE INVENTION

The following detailed description is of the best currently contemplated modes of carrying out exemplary embodiments of the invention. The description is not to be taken in a limiting sense, but is made merely for the purpose of illustrating the general principles of the invention, since the scope of the invention is best defined by the appended claims.

Broadly, an embodiment of the present invention provides a combination of four wound care products and a method: (1) a wound vacuum dressing to provide a more sterile cover over the wound to prevent infection, provide a closed system (air tight) to prevent maceration and soiling from drainage to the surrounding skin and peg insole pad, to introduce and maintain negative pressure, to decrease excessive fluid to the wound(s) and provide an environment to increase granulation and epithelialization; (2) a compression hose for additional support to the lower leg, ankle, and foot aiding in proper fluid pressure control in the lower extremity and protects against friction of the contact cast against the lower extremity; (3) a removable peg insole foot insert to offload pressure and friction from the wound as well as provide a pressure free pathway to the ankle connection point which prevents obstruction, and (4) a total contact cast with inner padding layer to further off load pressure from the wound(s) and vacuum dressing. Furthermore, a definite but adaptable method is disclosed outlined herein for application and configuration of the aforementioned four products such that all components function together to potentiate and aid in healing while remaining free from obstruction and allowing sufficient flexibility of design for the addition of desired or medically necessary customized dressing options, including but not limited to topical medications, ointment(s), and additional dressings for any wounds that may be present on the lower leg, ankle, or top of the foot. It should be noted that any and all such customizations should be applied in accordance with the methodology both in sequence and location such that non-interference with the proper placement and function of the wound vacuum, its connector, tubing, and fabric layer components are assured.

The vacuum cast, (“Vac-Cast”), can transfer weight bearing pressure from the bottom of the foot to the lower leg, significantly reducing positive pressure on the plantar wounds and vacuum dressing during ambulatory activity. The compression hose is needed to provide additional support to the lower leg, ankle, and foot aiding in proper fluid pressure control of the lower extremity and is needed to reduce friction between the contact cast and leg, ankle, and foot. The removable peg insole foot insert can further isolate the wound from ambulatory pressures and friction and redirect expected and unavoidable residual positive pressure from wound locations to non-injury areas on the bottom of the foot. The wound vacuum is used to create negative pressure on the wound, supply a sterile seal, and provide a pathway for drainage of fluids, fluid pressure, and edema. The surgical tubing from the vacuum dressing can be fitted with a Hemovac, Jackson-Pratt, or comparable vacuum and drainage device. The vacuum and drainage device can be attached to the top of the pressure cast for convenience.

Precise application methodology can ensure the proper function, potentiating, and interaction of all four components. The proper application, sequence, and configuration of the four wound care products listed herein encourages granulation and epithelialization and reduces the impacts of secondary conditions on clinical outcomes by creating a change in pressure conditions, and provides a sterile pathway for a vacuum and drainage device to maintain optimal fluid and fluid pressure conditions at the plantar wound(s) surface, and offers superior protection from non-sterile surfaces, friction, and the offloading of positive weight bearing pressures during ambulation. The method, sequence, and system of the present invention mitigate the risks inherent in the healing of plantar wounds for ambulatory patients, particularly those patients suffering from secondary conditions, and are directly responsible for rapid and dramatic improvements in healing as evidenced by multiple clinical trials on candidate patients.

The four products described above, in combination, using the described methodology and sequence herein, can improve healing rates and outcomes for the treatment of plantar wounds in ambulatory patients as evidenced by the outcomes of clinical trials and for the reasons conveyed above. This holds true for even the most challenging of chronic plantar wound cases including cases where amputation may be the prevailing prognosis.

The importance of the placement and configuration of the vacuum dressing connector and surgical tubing as described herein cannot be overstated. Proper configuration allows the products to function in combination as intended. Earlier attempts to combine the vacuum dressing and pressure cast malfunctioned because the designs attempted to route either the surgical tubing or the connector and vacuum dressing up the inside of the pressure cast and out the top of the cast. These configurations malfunctioned for the following reasons: (1) blockage of the surgical tubing occurred due to compression from the pressure cast, when tubing was run inside the cast and out the top rather than out the posterior ankle, and (2) the inner dressing fabrics failed to properly transmit pressure and fluids when the dressing was extended vertically inside the cast and out the top. Basically the physical forces required to create a vacuum and draw a fluid column vertically through fabric are orders of magnitude greater than when fabrics are terminated near the base and fluid and pressure are directed horizontally out through the pressure cast inside surgical tubing that remains unobstructed and relatively close to the base of the foot (the KCI bridge product in particular was found to malfunction in clinical trials when fabric extended vertically and/or inside the pressure cast). Earlier attempts to combine only the vacuum dressing with the removable peg sole insert and compression hose did not result in significant statistical improvements in healing rates because the potential of the removable peg sole insert to protect plantar wounds from the entirety of weight bearing loads was insufficient in the absence of the pressure cast component. It is also notable that some providers failed to properly extend the inner layers of the vacuum dressing between wound surface and connector. The vacuum cast will fail to function properly unless the vacuum dressing inner layers are extended continuously from underneath the wound to underneath the connector which enables the fabric to properly transport fluids and pressure through the system.

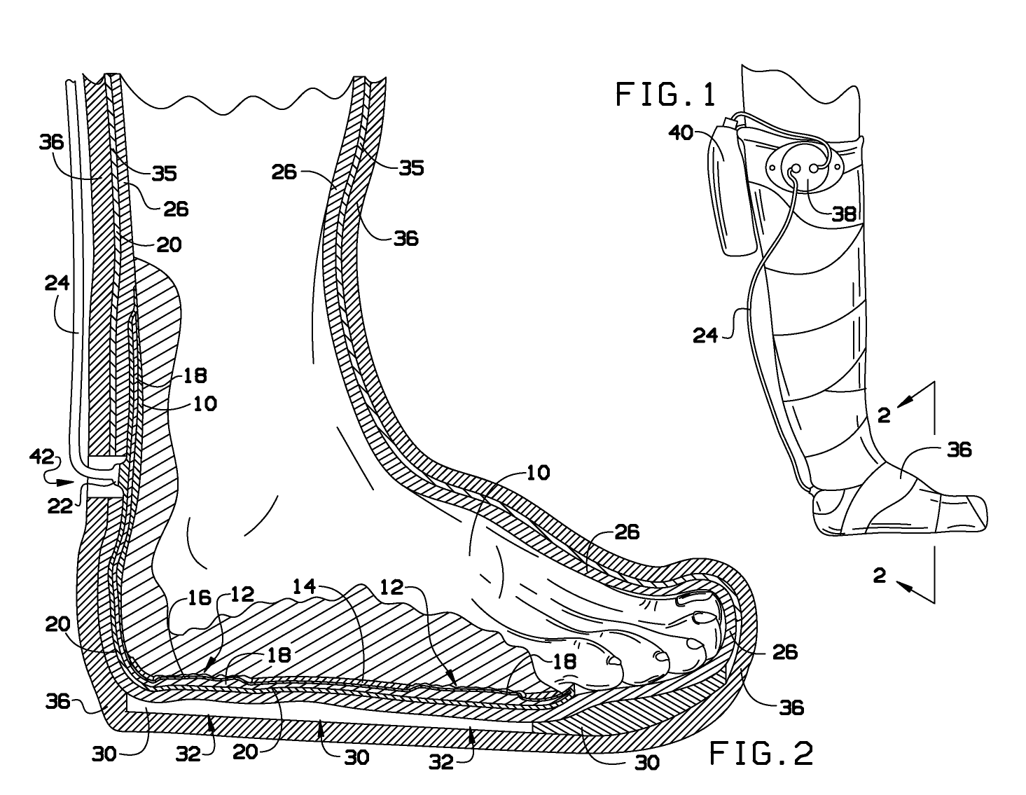

Referring now to FIGS. 1 and 2, FIG. 1 shows an overview of an applied vacuum cast according to an exemplary embodiment of the present invention, showing a pressure cast 36 as the exterior layer with an extraction tube 36 running to a vacuum device 38 that can be further connected to a drainage reservoir 40 for collecting drainage taken out of the wounds.

FIG. 2 shows a partially cut-away cross-sectional view of the vacuum cast of FIG. 1. The various layers will be described in their application with respect to the drawings below.

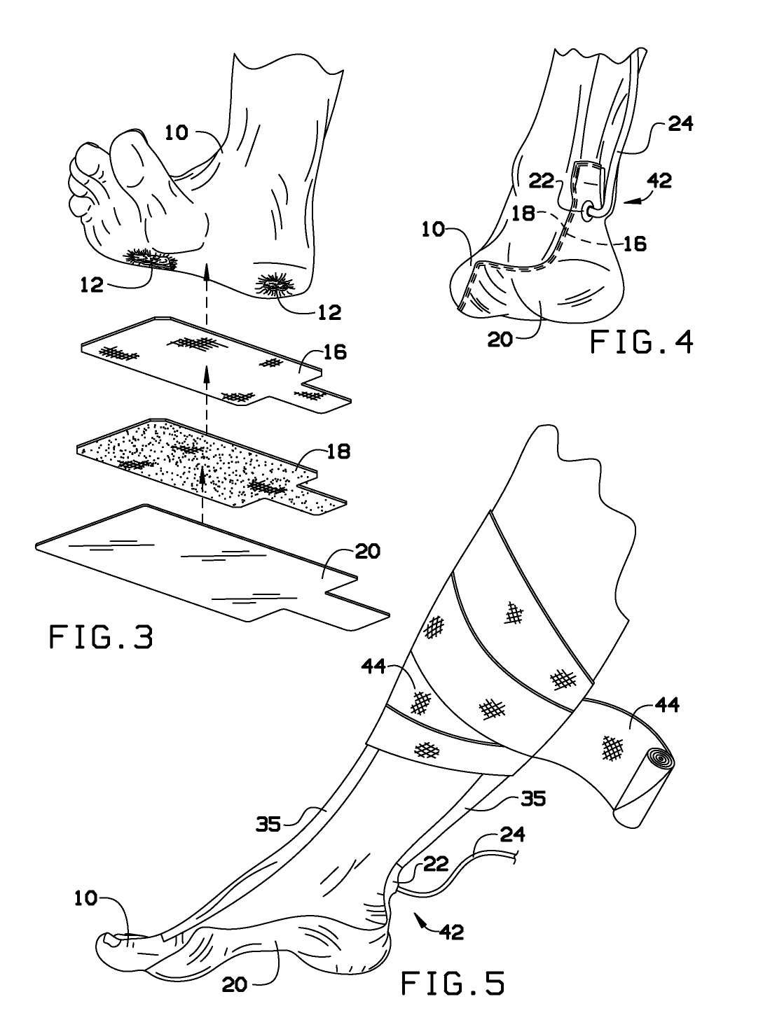

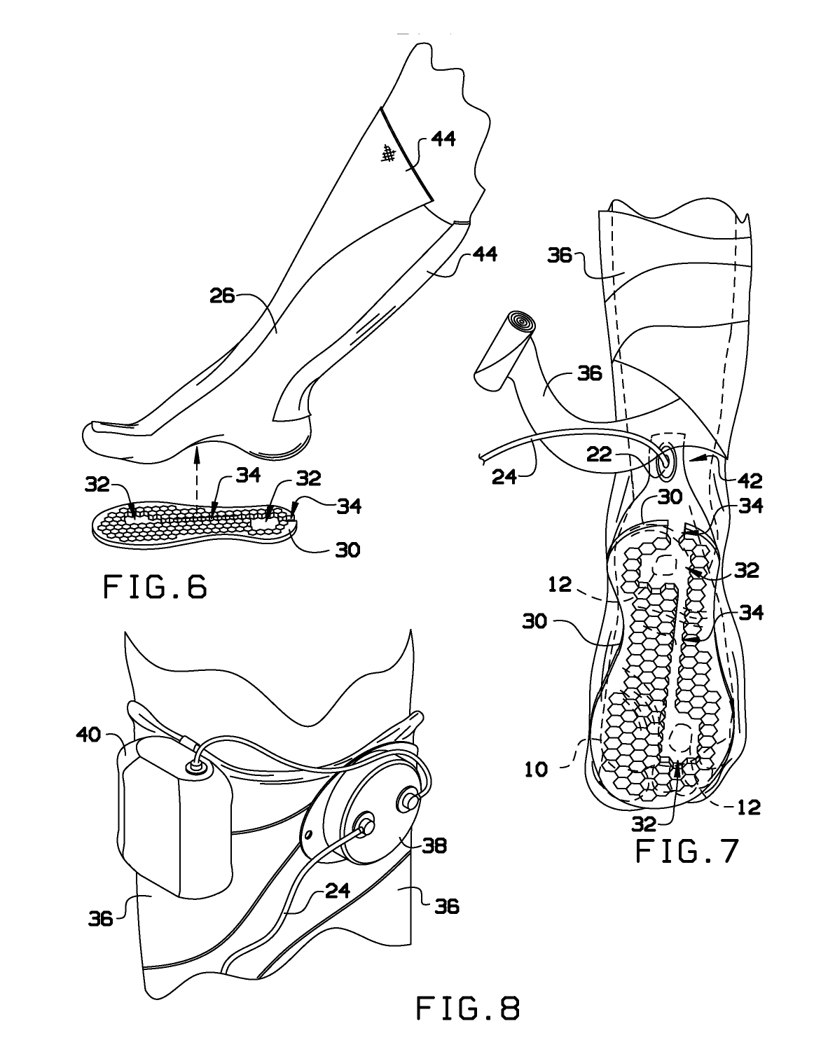

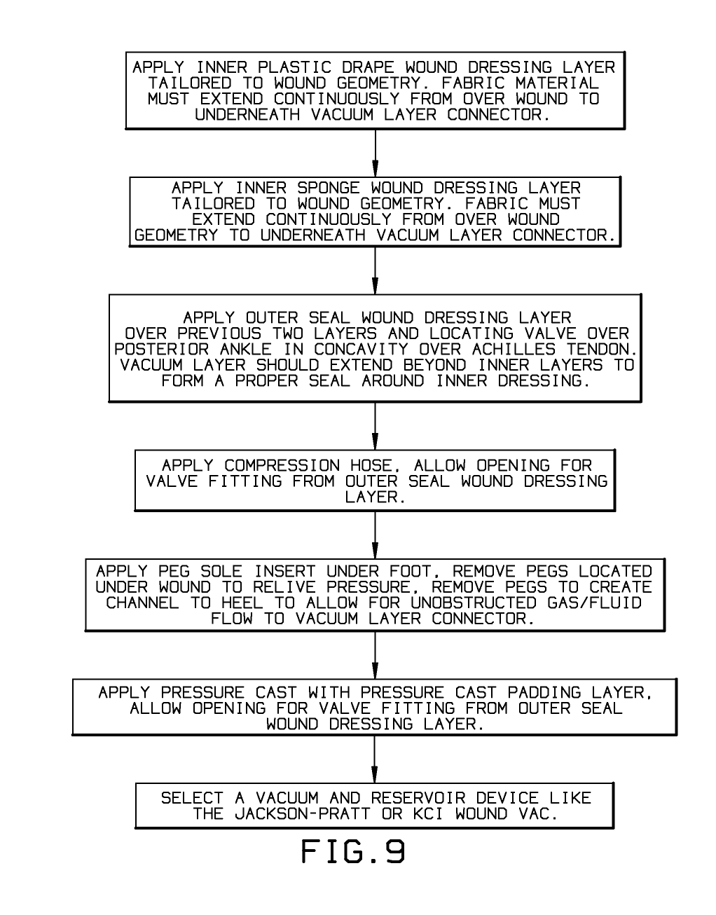

Referring now to FIGS. 3 through 9, an exploded perspective view showing the first step which is applying a wound vacuum dressing. FIG. 3 depicts two discreet locations of plantar wounds 12 on a patient's foot 10 for illustrative purposes; however, a single wound is more common. This design also works if a wound is present underneath the wound vacuum connector 22. The wound(s) 12 can be cleaned and any required and appropriate topical medications or ointments can be applied to the wound 12 and/or skin 14 prior to introduction of the vacuum (negative pressure) dressing. The dressing fabrics can be tailored to fit over the wound(s) geometry and to address variability in medical needs. The fabric layers include: 1) an inner plastic drape 16 (to prevent the patients skin from being in contact with the drainage from the wound) 2) an inner adsorptive layer 18, i.e., sponge (to move fluid away from the wound itself), and 3) an outer seal cover 20 (to make the system airtight so that negative pressure exists to remove the drainage and excess fluid). All fabric layers must cover the wound(s) surface area completely and extend continuously and contiguously from the wound(s) surface to the wound vacuum connector 22.

The wound vacuum connector 22 penetrates the outer seal cover layer 20 and seats above the inner drape 16 and sponge layers 18 and is typically located over the posterior ankle in the concavity over the Achilles tendon 42. The components can be configured such that the connector 22 and surgical tubing 24 are free from obstruction and therefore exits the total contact cast at the base (Achilles area). This design provides a continuous conduit for pressure and fluids to travel from the ulcer to the holding cell outside the total contact cast. This enables maximization of permeability for the inner dressing layers, enables the introduction and maintenance of negative pressure conditions, and prevents malfunction of the system when used in combination with the additional necessary components depicted in FIGS. 5 through 7. It should be noted that the insole with removable pegs not only offloads the wound itself, but also the pathway from the wound to the ankle.

Step 2 is the application of a compression stocking 26 over the wound vacuum dressing 20 extending from below the knee to just behind the toes. A hole can be cut in the compression stocking to allow for the connector 22 and surgical tubing 24 to pass through the compression stocking 26. The compression stocking 26 serves to reduce friction and further protect the skin from the cast and aids in fluid pressure support for the lower leg which decreases excess fluid pressure and prevents seepage out of the wound. It should be noted that the compression stocking 26 can be substituted with a regular non compression liner if the patient suffers from severe arterial insufficiency.

Step 3 is the application of a removable peg foot insole 30 which offloads pressure to the wound(s) when pegs are removed from under the wound(s) to form a peg sole wound cavity 32 to relieve pressure to the wound area 12 during ambulation and to clear a pressure free path 34 from the ulcer to the ankle. The insole 30 is applied outside the layered wound vacuum dressing from Step 1 and compression hose 26 from Step 2.

A pressure cast layer 44 includes two layers: 1) an outer hardened shell 36 (also referred to as pressure cast 36) and 2) an inner padded lining 35 to further protect the skin from friction and abrasions from the total contact cast. The pressure cast layer 44 can be placed over and around the calf, ankle, foot, over the compression hose 26 from Step 2, over the removable peg sole insole 30 from Step 3, and over the vacuum dressing 20 from Step 1.

FIG. 7 shows how the posterior side of the pressure cast layer 44 can be applied over the vacuum dressing, compression stocking, and the removable peg insert from Steps 1, 2, and 3. This perspective view also shows how the connection fitting and surgical tubing can be placed over the posterior ankle in the concave recess of the Achilles tendon and can extend through the compression hose and pressure cast by either making a hole in the cast before it hardens and cures or applying the pressure cast fabric around the connector and tubing if using the “wrap around the leg” method of casting. This step prevents compression of the connection or surgical tubing which can restrict pressure and fluid flow and cause the vacuum dressing to malfunction.

FIG. 8 depicts how the surgical tubing 24 is fitted with a vacuum device 38, such as a Hemovac, Jackson-Pratt like device, or other comparable vacuum device. It is important to also note that a traditional wound vacuum (such as the product offering by KCI) is not required unless the wound is particularly deep. The selected vacuum device 38 can then be attached to the top of the pressure cast 36 for convenience. A drainage reservoir 40 can be attached to the vacuum device 38 for collection of fluids. In some embodiments, the vacuum device 38 can be integrated with the drainage reservoir 40.

These steps are provided in greater detail below with respect to an exemplary embodiment of the present invention.

In an exemplary embodiment of the present invention, the Vacuum Cast involves the following steps. First, a multi layered wound vacuum dressing including the inner drape layer 16, and the inner sponge layer 18 can be tailored to the wound geometry, and an outer sterol sealing layer (vacuum seal layer 20) placed over the inner layers extending out over and beyond the inner dressings sufficient to insure a sterile outer vacuum seal. The vacuum dressing connector 22 can be formed in an extended flap in the outer vacuum seal layer 20. All inner and outer layers of the vacuum dressing must extend from over the area of the wound to underneath the area of the connector and surgical tubing forming a continuous and contiguous permeable conduit. The flap allows the connector to be placed above the patients heal and over the posterior ankle in the low profile concavity of the Achilles tendon 42, allowing the connector 22 and surgical tubing 24 to penetrate and protrude from the outer pressure cast 36 such that no compression of the components takes place which could cause a malfunction of the dressing. It should be noted that the dressing will function properly even if wounds are located underneath the connector.

Next, the compression hose 26, for additional support to the lower leg, ankle, and foot, to aid in proper fluid pressure control and reduce friction, can be added over the vacuum dressing extending from below the knee to just behind the toes. A hole should be cut in the compression hose 26 that is large enough to allow for the connector 22 and surgical tubing 24 from the vacuum dressing 20 to pass through un-impeded. The compression hose reduces friction, supports the leg, and helps to maintain fluid pressures.

Next, the removable peg insole 30 foot insert can be applied by removing pegs under and around the outside perimeter of the plantar wound(s) and in a line out to the connector to create a fluid channel and address the problems of residual positive pressure on the wound and dressing during ambulation. The peg insole 30 further isolates the wound from ambulatory pressures, friction, and directs positive pressure to non-injury areas on the bottom of the foot while protecting and preserving the negative pressure provided by the wound vacuum and further protecting wound and vacuum dressing from non-sterile surface contact.

Next, the contact cast can be applied such that the contact cast transfers significant weight baring pressure from the bottom of the foot to the lower leg, reducing positive pressure on the plantar wounds and vacuum cast during ambulatory activity. The contact cast can be applied in two layers with a hardened outer layer 36 and a protective padded inner layer 35 to protect the lower extremity from friction of the outer layer. The connector 22 and surgical tubing 24 from the vacuum dressing can protrude through the cast over the posterior ankle in the low profile concavity of the Achilles tendon such that no compression of the vacuum dressing components takes place which could cause a malfunction of the dressing

The vacuum and drainage functions can be provided by various devices. For example, a Hemovac or Jackson-Pratt drainage and vacuum device can be used. The vacuum device 38 can be attached to the top of the cast for easy access. A drainage reservoir 40 can be attached to the vacuum device 38 for collecting fluids and providing easy draining thereof.

It has been observed that ambulatory patients experience too much positive pressure, friction, and exposure to bacteria and other sources of infection on their plantar wound(s) during ambulatory movement. However, using the contact cast, compression hose, removable peg insole foot insert, and vacuum dressing in combination provides superior weight and pressure off-loading which protects against the positive pressures and friction of ambulation and preserves the negative pressures introduced by the vacuum dressing. This methodology and combination treatment substantially improves clinical outcomes and healing rates in even the most chronic of plantar wound cases particularly when patients are also suffering from secondary conditions such as venous insufficiency, circulatory and/or immune system deficiencies including diabetes. It should be noted that proper placement and configuration of the products insures that the vacuum dressing functions as designed and intended.

In some embodiments of the present invention, a kit can be produced by a qualified manufacturer. The kit can contain instructions, components and material for accomplishing all four of the above identified steps utilizing the above described methodology and instructions to insure proper location and configuration of the products and provide consistency in the intended function and performance. The kit can provide convenience, consistency, and time savings to the medical provider.

Physicians/providers are generally reluctant to engage in complex procedures particularly if they are unfamiliar with the process or components or if they cannot ensure consistency of quality or products. Time is also a factor in medical procedures and, by having a kit, the time to provide the service can be reduced. Having all four components provided in a single kit or class of kits provides the consistency that physicians want and reduces the time required for physicians to gather the components and provide the service.

Physicians also want to reduce liability and when there is variation in products or procedures (i.e., they have to select and combine the different products themselves), they run the risk of having to defend each selection decision and each treatment event as separate and distinct acts in the event of a law suit because it could be argued that there was significant or substantial variation in quality, product or component selection, or process variation between patient encounters and treatments. A kit can bring consistency of quality, repeatability, uniform process, and time savings to the equation.

It should be understood, of course, that the foregoing relates to exemplary embodiments of the invention and that modifications may be made without departing from the spirit and scope of the invention as set forth in the following claims.

What is claimed is:

1. A vacuum cast comprising:
a layered wound vacuum dressing applied to plantar wounds of a patient;
a vacuum connector applied to an outer sealed vacuum layer of the layered wound vacuum dressing;
extraction tubing extending from the vacuum tubing to a vacuum device;
a peg insole applied to a sole of a foot of the patient, the peg insole having one or more wound cavities corresponding to a location of the plantar wounds, and a channel interconnecting the one or more wound cavities with a posterior portion of the foot; and
a pressure cast layer applied about a lower leg and foot of the patient.

2. The vacuum cast of claim 1, wherein the layered wound vacuum dressing includes a drape layer, a sponge layer and the sealed vacuum layer with connector.

3. The vacuum cast of claim 1, wherein the layered wound vacuum dressing drape layer and sponge layer extend continuously from over wound to underneath connector.

4. The vacuum cast of claim 1, further comprising a compression hose disposed under the pressure cast layer.

5. The vacuum cast of claim 1, wherein the pressure cast layer includes a pressure cast padding and a pressure cast.

6. The vacuum cast of claim 1, further comprising a vacuum device for drawing out fluids and pressure from the wound site and through the extraction tubing.

7. The vacuum cast of claim 1, further comprising a fluid drainage holding cell for collecting fluids drawn through the extraction tubing by the vacuum device.

8. The vacuum cast of claim 1, wherein the vacuum connector is disposed in an Achilles heel concavity region of the patient.

9. A method for protecting and encouraging healing of plantar wounds, the method comprising:
applying a layered wound vacuum dressing to plantar wounds of a patient, to reduce pressure, introduce negative pressure, and protect from friction, exposure to bacteria, and provide a conduit for the drainage of fluid and fluid pressure;
locating and positioning a connector and extraction tubing to a sealed vacuum layer of the layered wound vacuum dressing, the connector being located over a posterior ankle in a low profile concavity of the Achilles tendon such that no compression of the connector and extraction tubing takes place;
extending all inner and outer layers of the layered wound vacuum dressing from over an area of the wound to underneath an area of the connector and extraction tubing, forming a continuous and contiguous permeable conduit;
applying a compression hose over a lower leg and the layered wound vacuum dressing, starting from below a knee and extending to just behind toes of the patient;
applying an adjustable foot sole insole on a bottom of the foot of the patient, the insole having one or more wound cavities for additional offloading and pressure redirection;
applying a pressure cast layer around the calf, ankle, and foot of the patient, the pressure cast layer covering the layered wound vacuum dressing and the insole to off load weight bearing pressure while allowing the connector and extraction tubing to protrude and project through the pressure cast layer un-impinged; and
applying a vacuum device to the surgical tubing to create and maintain negative pressure on the wound and provide a conduit for removal of fluids and fluid pressure.

8. The method of claim 7, wherein the layered wound vacuum dressing includes drape and sponge inner layers and a vacuum seal sterile outer layer.

9. The method of claim 7, further comprising making a hole in the compression hose for the connector and extraction tubing of the vacuum dressing to pass through.

10. The method of claim 7, wherein the pressure cast includes an outer rigid shell and inner padding layer.
